(19) United States
(12) Patent Application Publication
(10) Pub. No.: US 2015/0374149 A1
(43) Pub. Date: Dec. 31, 2015

(54) SLEEP AIDING APPARATUS AND USES THEREOF

(71) Applicant: Chang Gung Memorial Hospital, Taoyuan, Taoyuan (TW)

(72) Inventor: May-Kuen Kuam Wong, GUISHAN (TW)

(21) Appl. No.: 14/685,940

(22) Filed: Apr. 14, 2015

Related U.S. Application Data
(60) Provisional application No. 62/018,974, filed on Jun. 30, 2014.
(60) Provisional application No. 62/064,622, filed on Oct. 16, 2014.

(51) Int. Cl. A47G 9/10; A61M 21/02

(52) U.S. Cl. CPC A47G 9/1045; A61M 21/02; A47G 2009/006

(57) ABSTRACT

A sleep aiding apparatus for increasing the duration of deep sleep and/or reducing the sleep latency of a subject is disclosed. The sleep aiding apparatus comprises a pillow body and a device to play a sleep aiding music. Also disclosed herein are methods for aiding sleep, by providing the sleep aiding apparatus to a subject in need thereof.

### CROSS-REFERENCE TO RELATED APPLICATIONS

This application claims the benefit of U.S. Patent Application No. 62/018,974, filed on Jun. 30, 2014, and U.S. Patent Application No. 62/064,622, filed on 16 Oct., 2014, the disclosure of all of which are incorporated by reference herein in their entirety.

### BACKGROUND OF THE INVENTION

Human spend roughly a third of their life sleeping, which helps to restore physical stamina and energy. Good-quality sleep, especially deep sleep, is essential for body to repair, tissue to regenerate and immune system to strengthen. Altered sleep-wake cycles lead to fatigue, reduced concentration, difficulty falling asleep, and loss of appetite. In addition, the duration of deep sleep reduces with aging.

There is still a need for sleep-improvement strategy to improve sleep quality, such as increasing the duration of deep sleep, for patients experiencing sleep disorders and for relatively healthy people who have short-term sleep problems caused by specific events. The present invention addresses this need.

### BRIEF SUMMARY OF THE INVENTION

Some embodiments provide a sleep aiding apparatus, including a pillow body and a device which plays a sleep aiding music having a regular rhythm of about 50 to about 70 beats per minute, wherein the sleep aiding apparatus increases the duration of deep sleep or reduces sleep latency. In one embodiment, the increase of duration of deep sleep or reduction of sleep latency is relative to a sleep aiding apparatus without a sleep aiding music having a regular rhythm of about 50 to about 70 beats per minute. In another embodiment, the increase of duration of deep sleep or reduction of sleep latency is relative to a pillow body with the height of the substantially convex portion of less than about 7 cm or greater than about 13.5 cm.

In one embodiment, the pillow comprises a top surface, a bottom surface, and at least on edge surface, wherein the top surface comprises a substantially concave portion for head resting and a substantially convex portion adjacent to the substantially concave portion for neck supporting, and the height of the substantially convex portion is about 7 to about 13.5 cm.

Some embodiments are directed to methods to aid sleep in a subject in need thereof, comprising, providing a pillow body described herein and a sleep aiding music to the subject, wherein the sleep aiding music has a regular rhythm of about 50 to about 70 beats per minute, and the subject's deep sleep duration increases or sleep latency reduces. In one embodiment, the increase of duration of deep sleep or reduction of sleep latency is relative to a sleep aiding apparatus without a sleep aiding music having a regular rhythm of about 50 to about 70 beats per minute. In another embodiment, the increase of duration of deep sleep or reduction of sleep latency is relative to a pillow body with the height of the substantially convex portion of less than about 7 cm or greater than about 13.5 cm.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### BRIEF DESCRIPTION OF THE DRAWINGS

Illustrative embodiments of the present invention are described in detail below with reference to the following Figures:

FIG. 1A is an angled top view of an embodiment of the sleep aiding apparatus of the present invention.

FIG. 1B is a cross sectional view of an embodiment of a sleep aiding apparatus of the present invention.

FIG. 2A is a cross section view of a subject in supine position while using of an embodiment of the sleep aiding apparatus of the present invention.

FIG. 2B is a cross section view of a subject in side-lying position while using an embodiment of the sleep aiding apparatus of the present invention.

FIG. 2C is a schematic top view of a subject in supine position while using an embodiment of the sleep aiding apparatus of the present invention.

### DETAILED DESCRIPTION OF THE INVENTION

As employed above and throughout the disclosure, the following terms, unless otherwise indicated, shall be understood to have the following meanings.

### DEFINITIONS

As used herein, the singular forms “a,” “an,” and “the” include the plural reference unless the context clearly indicates otherwise.

The term “subject” as used herein typically refers to a human or an animal subjected to the methods described herein. It is to be understood that a subject can be a patient with known or suspected sleep disorder, but subjects without known or suspected sleep disorder, such as research subjects, are also included within the scope of the term “subject.”

All numbers herein may be understood as modified by “about.”

When an element or layer is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. For the purposes of this disclosure, “at least one of X, Y, and Z” may be construed as X only, Y only, Z only, or any combination of two or more of X, Y, and Z, such as, for instance, XYZ, XYY, YZ, and ZZ.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and will not be interpreted in an idealized or overly formal sense, unless expressly so defined herein.

### Sleep Aiding Apparatus

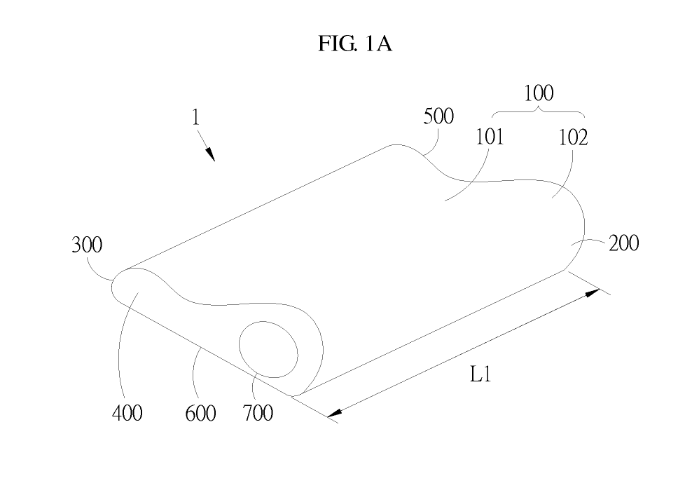

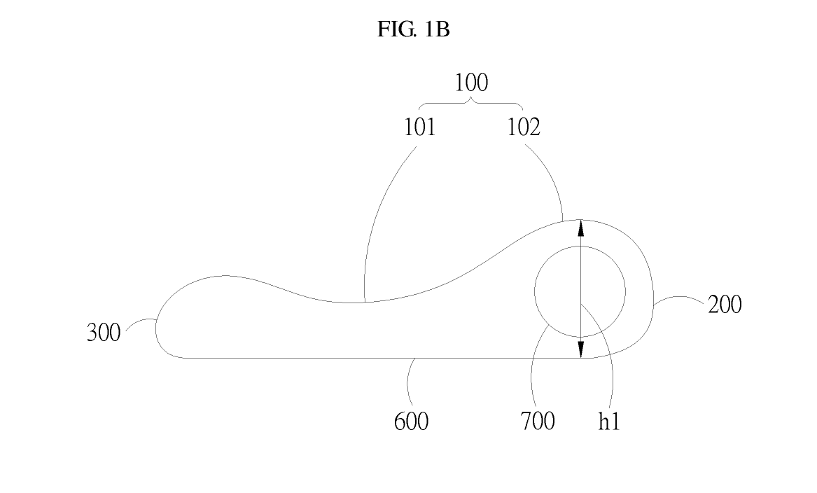

Referring to FIG. 1A and FIG. 1B, the present invention provides a sleep aiding apparatus includes a pillow body 1 and a device 700 for playing sleep aiding music. In one embodiment, the sleep aiding apparatus increases the subject's deep sleep duration. In another embodiment, the sleep aiding apparatus reduces the subject's sleep latency.

In one exemplary embodiment, the device 700 is connected to the pillow body 1. As illustrated in FIG. 1A and FIG. 1B, one embodiment provides a device 700 with one or more speakers embedded in the left edge surface 400 and/or in the right edge surface 500 to ensure the subject listens to the sleep aiding music while using the pillow body 1. In another exemplary embodiment, the device 700 is not connected to the pillow body 1. In yet another exemplary embodiment, the pillow body 1 including a top surface 100, a bottom surface 600, at least one edge surface, such as the front edge surface 200, a back edge surface 300, a left edge surface 400, and a right edge surface 500. The top surface 100 comprises a substantially concave portion 101 for resting the head of a subject and a substantially convex portion 102 adjacent to the substantially concave portion 101 for supporting the neck of a subject. The maximum height h1 of the plateau of the substantially convex portion 102 is about 7 to about 13.5 cm. In an exemplary embodiment, the maximum height h1 of the plateau of the substantially convex portion 102 is equal to or less than about 13 cm, 12.5 cm, 12 cm, 11.5 cm 11 cm, 10.5 cm, 10 cm, 9.5 cm, 9 cm, 8.5 cm, 8 cm, 7.5 cm or any value or range of values therebetween in 0.1 cm increments (e.g., about 9.3 cm, about 10.9 cm, about 7.7 cm to about 10.6 cm, etc.). In one exemplary embodiment, the maximum height h1 of the plateau of the substantially convex portion 102 is about 9 cm to about 11.5 cm. In another exemplary embodiment, the maximum height h1 of the plateau of the substantially convex portion 102 is about 10 cm to about 11 cm.

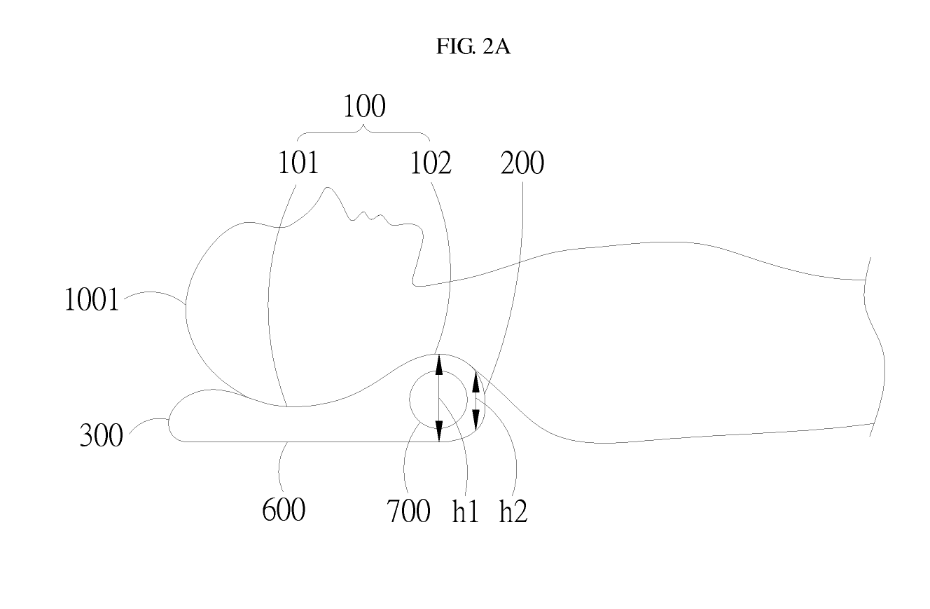

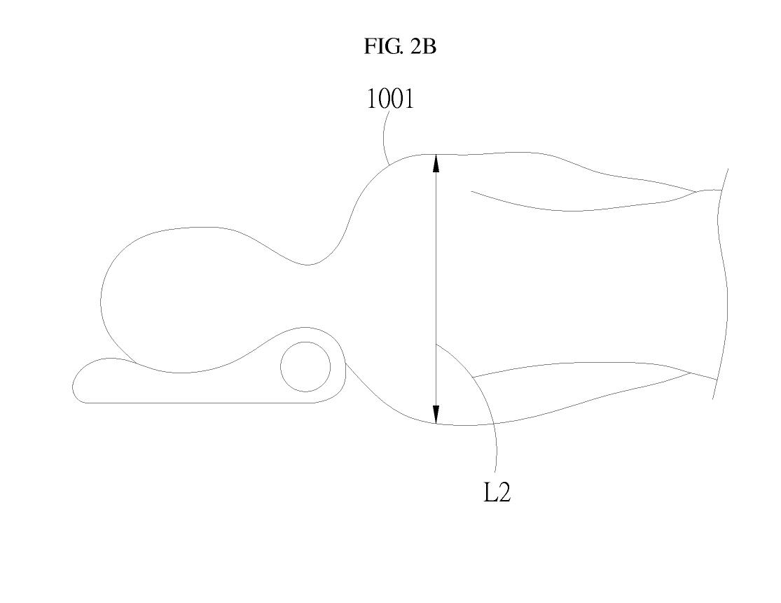

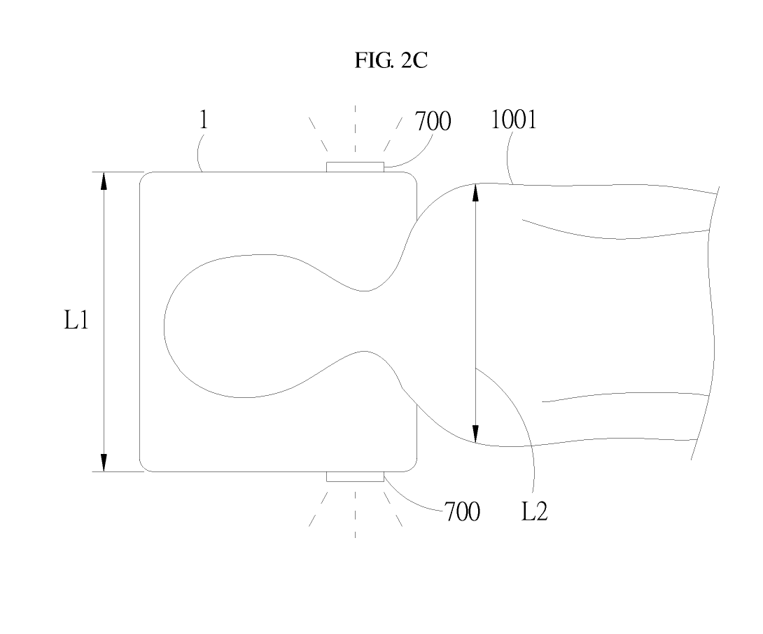

In one embodiment, the maximum height h1 of the substantially convex portion 102 is higher than the height h2 of the front edge surface 200 in FIG. 2A. In an embodiment, the height h2 of the front edge surface 200 is equal to or less than about 8 cm, 7.5 cm, 7 cm, 6.5 cm 6 cm, 5.5 cm, 5 cm, 4.5 cm, 4 cm, or any value or range of values therebetween in 0.1 cm increments (e.g., about 5.3 cm, about 7.9 cm, about 6.2 cm to about 7.2 cm, etc.).

In another embodiment, the height h1 of the substantially convex portion 102 may be lower than the height h2 of the front edge surface 200.

Referring to FIG. 1A and FIG. 2C, the front edge surface 200 has a length L1, wherein L1 is longer than the subject's shoulder length L2. In one embodiment, the shoulder length L2 is determined by measuring the arc distance from the left (right) acromion through the back neck point to the right (left) acromion

In one embodiment, the pillow body 1 is substantially free of any grooves for hand rest.

The sleep aiding music has a regular rhythm and smooth melody. In one embodiment, sleep aiding music has a regular rhythm of about 50 to about 70 beats per minute. In another embodiment, the sleep aiding music has a regular rhythm of about 55 to about 65 beats per minute. In yet another embodiment, the sleep aiding music has a regular rhythm of about 60 beats per minute. In yet another embodiment, the sleep aiding music has a regular rhythm of about 51, 52, 53, 54, 55, 56, 57, 58, 59, 60, 61, 62, 63, 64, 65, 66, 67, 68 or 69 beats per minute.

The device 700 is a playback device or any type of media player. Non limiting examples of the playback device include compact disc, DVD player, MP3 player, tape recorder, speaker, earphone, and mobile communication device.

As shown in FIG. 2A to FIG. 2C, the substantially convex portion 102 of the pillow body 1 provides a neck support for the subject 1001, to prevent misalignment of the subject's cervical spine or neck.

The pillow body 1 comprises one or more materials selected from the following: foam, inflatable bladder, cotton, down, feathers, buckwheat hulls, hemp fill, latex, or polyester. The material in the pillow body is sufficiently stiff to maintain height L1 between about 7 cm to about 13.5 cm while the subject's head is on the pillow body 1.

### Methods for Aiding Sleep

Another aspect of the present invention is directed to methods for aiding sleep, comprising (a) providing a pillow body 1 described herein to a subject in need thereof, and (b) providing a sleep aiding music to the subject, wherein the sleep aiding music has a regular rhythm of about 50 to about 70 beats per minute. In one embodiment, the method increases the subject's deep sleep duration. In another embodiment, the method reduces the subject's sleep latency.

Combination of a pillow body 1 with a convex portion height h1 of about 7 to about 13.5 cm and a sleep aiding music with a regular rhythm of about 50 to about 70 beats per minute synergistically increases the subject's deep sleep duration and/or reduces the subject's sleep latency, relative to a sleep aiding apparatus without a sleep aiding music having a regular rhythm of about 50 to about 70 beats per minute or relative to a pillow body with a convex portion height h1 of less than about 7 cm or greater than about 13.5 cm. The increase of deep sleep duration, such as Stage III sleep, restores the body function and lead to a better physical and mental well-being.

Normal human non-rapid eye movement (Non-REM) sleep is composed of four stages (I, II, III, and IV) ranging from the lightest to the deepest sleep. Stage I sleep is the transition from wakefulness to light sleep and is the lightest stage of sleep. Stage II sleep constitutes up to 50% of total sleep time and is a true physiologic stage of sleep. Stages III and IV sleep are the deep sleep stage with slow brain waves (called delta wave) on EEG reading.

The different stages of sleep (stage 1-4) can be determined by methods well known in the art, such as polysomnography (PSG) or EEG rhythms. For example, stage I sleep is characterized by the disappearance of alpha wave and appearance of theta wave, stage II sleep is characterized by the presence of k complexes and sleep spindles, stage III sleep is characterized by the appearance of delta wave and stage IV sleep is characterized by the continuation of delta wave.

Embodiments of the present invention are illustrated by the following examples, which are not to be construed in any way as imposing limitations upon the scope thereof. During the studies described in the following examples, conventional procedures were followed, unless otherwise stated. Some of the procedures are described below for illustrative purpose.

### Example 1

### Clinical Evaluation of the Sleep Aiding Apparatus on Sleep Parameters

Recruitment Protocol:

A young female subject, without (a) major depressive disorders; (b) auditory dysfunction; (c) sleep disorders, such as insomnia, narcolepsy, snoring, sleep apnea, or periodic limb movement syndrome; (d) use of medications that influences sleep, such as hypnotics, sedatives, antidepressants, anticholinergics, antihistamines, tranquilizers, or melatonin; (e) a habit of meditation or relaxation in the evening within 30 minutes of bedtime; (f) a habit of not going to bed until midnight; and (g) difficulty falling asleep in an unaccustomed environment, was recruited for the sleep study, to evaluate the effect of one embodiment of the sleep aiding apparatus of the present invention on sleep parameters.

Sleep Study Protocol:

The sleep aiding apparatus, as illustrated in FIG. 1A, was used in this sleep study.

Polysomnography (PSG) was performed using the Compumedics P-series PS2 (Compumedics, Victoria, Australia) system to assess sleep parameters. PSG assessments were done according to the standard procedure recommended by the American Academy of Sleep Medicine. The sleep study was conducted between 6:30 pm to 8:30 pm on each testing day. The subject slept in the same room throughout the study and the room temperature was set at 25° C.

The female subject spent one night in each of the following 5 settings:
Setting 1: the study subject rested her head on a pillow body 1, wherein the height of the plateau (h1) of the substantially concave portion of the pillow body 1 was about 6 cm. There was no music during the study.
Setting 2: the study subject rested her head on a pillow body 1, wherein the height of the plateau (h1) of the substantially concave portion of the pillow body 1 was about 18 cm. There was no music during the study.
Setting 3: the study subject rested her head on a pillow body 1 of the present invention, wherein the height of the plateau (h1) of the substantially concave portion of the pillow body 1 was about 10 cm. There was no music during the study
Setting 4: the study subject rested her head on the pillow body 1 of the present invention, wherein the height of the plateau of the substantially concave portion of the pillow body 1 was about 10 cm. A sleep aiding music, with an irregular rhythm was played throughout the study.
Setting 5: the female subject rested her head on the pillow body of the present invention, wherein the height of the plateau of the substantially concave portion of the pillow body was about 10 cm. A sleep aiding music of the present invention, with a regular rhythm of about 60 beats per minute, was played throughout the study.

The female subject was hooked up to the PSG recording sensors to measure the following outcomes:
1. 3 non-REM sleep stages (Stage I-III) based on PSG recording,
2. Sleep latency: the length of time that takes to accomplish the transition from full wakefulness to stage 1 sleep, based on EEG reading.
3. Sleep efficiency: the number of minutes of sleep divided by the number of minutes in bed.
4. Hear rate (beats per minute)

Table I shows the results of the sleep study:

TABLE I

| | Setting | | | | |
|---|---|---|---|---|---|
| | 1 | 2 | 3 | 4 | 5 |
| sleep latency (min) | 8.5 | 5.5 | 7 | 9 | 6 |
| Duration of Each Sleep Stage in % | | | | | |
| Stage I | 36.5 | 37.5 | 12.3 | 20.4 | 11.5 |
| Stage II | 50.9 | 55.4 | 52.2 | 76.5 | 55.6 |
| Stage III | 2.5 | 7.1 | 12.3 | 3.1 | 24.4 |
| Sleep Efficiency (%) | 54.8 | 48.3 | 90.5 | 77.8 | 89.7 |
| Heart Rate (beats/min) | 72 | 59 | 61 | 62 | 60 |

Results: The present study shows that the pillow of the present invention (with the convex portion height of about 10 cm, setting 3) was associated with an increase in the percentage of stage III sleep or deep sleep (12.3%) compare to that of a pillow with a convex portion height of about 6 cm (2.5%) in setting 1 and a convex portion height of about 18 cm (7.1%) in setting 2. Similarly, the pillow of the present invention (with the convex portion height of about 10 cm, setting 3) was associated with an increase in sleep efficiency (90.5%) compare to that of a pillow with a convex portion height of about 6 cm (54.8%) in setting 1 and a convex portion height of about 18 cm (48.3%) in setting 2.

The use of the pillow of the present invention (with a convex portion height of about 10 cm) in combination with a commercially available sleep aiding music (with an irregular rhythm) reduces the percentage of stage III sleep from 12.3% (setting 3, without music) to 3.1% (setting 4), a reduction in sleep efficiency from 90.5% (setting 3, without music) to 77.8% (setting 4) and an increase in sleep latency from 7 minutes (setting 3, without music) to 9 minutes (setting 4)

The use of the pillow of the present invention (with a convex portion height of about 10 cm) in combination with the sleep aiding music of the present invent (with a regular rhythm of about 60 beats/min) further increases the percentage of stage III sleep from 12.3% (setting 3, without music) to 24.4% (setting 5), reduces sleep latency from 7 minutes (setting 3, without music) to 6 minutes (setting 5) and a comparable sleep efficiency.

These results show that the convex portion height of the pillow plays an important role in sleep efficiency and duration of deep sleep. A sleep aiding music with a regular rhythm further increases the duration of stage III sleep, reduces sleep latency, without adversely affect sleep efficiency.

### Example 2

### Clinical Evaluation of the Sleep Aiding Music on Sleep Parameters

24 Young adult students (11 male and 13 female, mean age 24.5+2.6 years) were recruited for the sleep study, according to the recruitment protocol in Example 1.

The sleep study was carried out according to the sleep study protocol in Example 1, except the sleep study was carried out in 3 consecutive nights. During the first night of adaptation, the subject was hooked up to the recording sensors and asked to sleep according to the usual sleep schedule, without any intervention. The goal of this adaption night was to allow participants to get accustomed to the sleep laboratory environment. During the following two nights, each participant spent one night listening to the sleep aiding music of the present invention (having a regular rhythm of 60 beats per minute) for 1 hour and another night in the control (without music) condition. The subjects were allowed to sleep uninterrupted for the reminder of the night.

Table 2 shows the duration of each sleep stage between the music group and control group.

TABLE 2

| | | Control | |
|---|---|---|---|
| Sleep stage | Music | (without music) | p-Value |
| Stage I (min) | | | |
| Short SL | 12.7 ± 10.3 | 19.6 ± 32.0 | Pc = 0.20 |
| group (n = 12) | | | |
| Long SL | 6.8 ± 7.6 | 9.8 ± 10.2 | Pg = 0.23 |
| group (n = 12) | | | |
| | | | Pi = 0.60 |
| Stage II (min) | | | |
| Short SL group | 171.3 ± 42.3 | 188.0 ± 30.3 | Pc = 0.03* |
| Long SL group | 151.5 ± 35.2 | 166.1 ± 22.7 | Pg = 0.10 |
| | | | Pi = 0.87 |
| Stage III (min) | | | |
| Short SL group | 48.1 ± 18.8 | 48.3 ± 20.2 | Pc = 0.10 |
| Long SL group | 54.8 ± 14.8 | 44.9 ± 14.3 | Pg = 0.80 |
| | | | Pi = 0.85 |
| Stage IV (min) | | | |
| Short SL group | 30.3 ± 27.0 | 35.3 ± 24.9 | Pc = 0.50 |
| Long SL group | 49.8 ± 23.0 | 39.0 ± 24.8 | Pg = 0.22 |
| | | | Pi = 0.07 |
| Stages III + IV (min) | | | |
| Short SL group | 78.3 ± 36.1 | 83.5 ± 37.8 | Pc = 0.15 |
| Long SL group | 104.6 ± 22.7 | 84.0 ± 26.9 | Pg = 0.27 |
| | | | Pi = 0.02* |
| REM sleep (min) | | | |
| Short SL group | 71.4 ± 54.3 | 45.3 ± 20.4 | Pc = 0.27 |
| Long SL group | 50.9 ± 27.9 | 57.8 ± 22.3 | Pg = 0.72 |
| | | | Pi = 0.07 |

Values are expressed as mean ± standard deviation.
*p < 0.05
Pc, Pg, and Pi, p-value corresponding to condition and group main effects and interaction effect, respectively;
REM, rapid eye movement;
SL, sleep latency
Short SL group = Subjects with sleep latencies shorter than 10 minutes
Long SL group = Subjects with sleep latencies longer than 10 minutes

The results show that sleep aiding music with a regular rhythm of about 60 beats per minute was associated with an increase in stage III+IV and reciprocal decrease in stage II sleep in the long sleep latency group.

### Example 3

### The Use of Anthropometric Measurements to Predict Preferred Pillow Height in the Young and Elderly Subjects

Method:

60 healthy young (18-30 y/o) subjects (30 males and 30 females) and 39 old (60-80 y/o) subjects (19 males and 20 females) were recruited for this study. For each subject, the following anthropometric parameters were measured using manual tape method:
1. Head girth was measured as the circumference from the glabella through the opistocranion to the glabella;
2. Head side girth was determined as the circumference of the bitragion;
3. Head front girth was the distance between the glabella and the external occipital protuberance;
4. Neck girth was determined as the circumference of the neck, measured at the 4th cervical vertebra (C4);
5. head length was measured from the neck base to the top of the head;
6. Shoulder length was determined by measuring the arc distance from the left (right) acromion through the back neck point (BNP) to the right (left) acromion;
7. The half shoulder length was the distance from the lateral point of the neck base to one side of the acromion;
8. The bi-deltoid breadth was the maximum horizontal breadth between two deltoids (5 cm below the acromion)

Subjects then laid on supine position with a height adjustable pillow support for 10 minutes, for which the height was constantly adjusted until the study subject reported a maximal perceived comfort in 0.5 cm steps. After each adjustment, subjects were asked if current pillow height was more comfortable than the previous pillow height. If so, the pillow was fine-tuned up or down in 0.5 cm increments until the subjects felt pillow height was optimized. The preferred pillow height was measured at two summations.

Results:

For the supine posture, the preferred pillow height for the young and old groups were 9.7±1.5 and 10.5±2.1 cm, respectively (group difference, p=0.83. For side-lying posture, the preferred pillow height for the young and old groups were 10.4±1.5 and 10.4±2.1 cm, respectively (group difference, p=0.1). The linear prediction model showed that the preferred pillow height in the supine and side-lying position were 9 to 11 and 9 to 11.5 cm, respectively. Since the one side of neck to shoulder width averaged across all subjects was 11.8±1.4 cm, half shoulder length+1 cm is a preferred pillow height for most healthy people (just one side of neck to shoulder). The preferred height did not differ between age groups, between subject genders, or between supine and side-lying postures. As for anthropometric factors, the bi-deltoid shoulder breadth and body weight were found to account for the preferred pillow height.

### CONCLUSIONS

Preferred pillow height is affected by bi-deltoid shoulder breadth and body weight but not by age group, gender, or supine or side-lying postures. After normalization, the linear prediction model showed that the preferred pillow height in the supine and side-lying position was 9-11 cm and 9-11.5 cm, respectively.

All publications, patents, and patent applications cited in this specification are herein incorporated by reference as if each individual publication or patent application was specifically and individually indicated to be incorporated by reference.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

What is claimed is:

1. A sleep aiding apparatus to aid a subject to sleep, comprising
a pillow body, comprising:
a bottom surface;
a top surface comprising a substantially concave portion for head resting and a substantially convex portion adjacent to the substantially concave portion for neck supporting, wherein the height of the substantially convex portion is about 7 to about 13.5 cm;
at least one edge surface, and
a device plays a sleep aiding music having a regular rhythm of about 50 to about 70 beats per minute,
wherein the sleep aiding apparatus increases the duration of deep sleep or reduces the sleep latency of the subject.

2. The sleep aiding apparatus of claim 1, wherein the substantially convex portion prevents misalignment of the subject's cervical spine.

3. The sleep aiding apparatus of claim 1, wherein the height of the substantially convex portion is about 9 cm to about 11.5 cm.

4. The sleep aiding apparatus of claim 1, wherein the height of the substantially convex portion is about 10 to about 11 cm.

5. The sleep aiding apparatus of claim 1, wherein one of the edge surfaces is a front edge surface having a height of about 4.5 to about 8.5 cm.

6. The sleep aiding apparatus of claim 1, wherein the sleep aiding music has a regular rhythm of about 55 beats to 65 beats per minute.

7. The sleep aiding apparatus of claim 1, wherein the sleep aiding music has a regular rhythm of about 60 beats per minute.

8. The sleep aiding apparatus of claim 1, wherein the device is a playback device selected from compact disc, DVD player, MP3 player, tape recorder or mobile communication device.

9. The sleep aiding apparatus of claim 1, wherein the pillow body comprises one or more of the following: foam, inflatable bladder, cotton, down, feathers, buckwheat hulls, hemp fill, latex, or polyester.

10. The sleep aiding apparatus of claim 1, wherein one of the edge surfaces is a front edge surface having a length that is longer than the subject's shoulder length.

11. A method to aid sleep, comprising:
(a) providing a pillow body to a subject in need thereof, said pillow comprising:
a bottom surface;
a top surface comprising a substantially concave portion for head resting and a substantially convex portion adjacent to the substantially concave portion for neck support, wherein the height of the substantially convex portion is about 7 to about 13.5 cm;
at least one edge surface; and
(b) providing a sleep aiding music to the subject, wherein the sleep aiding music has a regular rhythm of about 50 to about 70 beats per minute,
wherein the subject's deep sleep duration increases or sleep latency reduces.

12. The method of claim 11, wherein the substantially convex portion prevents misalignment of the subject's cervical spine.

13. The method of claim 11, wherein the height of the substantially convex portion is about 9 cm to about 11.5 cm.

14. The method of claim 11, wherein the height of the substantially convex portion is about 10 to about 11 cm.

15. The method of claim 11, wherein one of the edge surfaces is a front edge surface having a height of about 4.5 to about 8.5 cm.

16. The sleep aiding apparatus of claim 11, wherein the sleep aiding music has a regular rhythm of about 55 beats to 65 beats per minute.

17. The method of claim 11, wherein the sleep aiding music has a regular rhythm of about 60 beats per minute.

18. The method of claim 11, wherein the playback device is a compact disc, a DVD player, an MP3 player, a tape recorder, or mobile communication device.

19. The method of claim 11, wherein the pillow body comprises one or more of the following: foam, inflatable bladder, cotton, down, feathers, buckwheat hulls, hemp fill, latex, or polyester.

20. The method of claim 11, wherein one of the edge surfaces is a front edge surface having a length that is longer than the subject's shoulder length.

21. The method of claim 11, wherein the sleep aiding music is provided by a device selected from compact disc, DVD player, MP3 player, tape recorder or mobile communication device.

22. The method of claim 11, wherein the device is in contact with the pillow body.
